(19) United States
(12) Patent Application Publication
(10) Pub. No.: US 2026/0325008 A1
(43) Pub. Date: Oct. 22, 2026

(54) NON-CONTACT PELVIC FLOOR ASSESSMENT AND TRAINING METHOD

(71) Applicant: Medlander Medical Technology Inc., Nanjing, Jiangsu (CN)

(72) Inventors: Zhangyin Ma, Nanjing, Jiangsu (CN); Zhiwei Lv, Nanjing, Jiangsu (CN); Zhengyang Bi, Nanjing, Jiangsu (CN); Miao Li, Nanjing, Jiangsu (CN)

(73) Assignee: Medlander Medical Technology Inc., Nanjing, Jiangsu (CN)

(21) Appl. No.: 19/114,134

(22) PCT Filed: Jun. 7, 2023
(86) PCT No.: PCT/CN2023/098803

§ 371 Date: Mar. 21, 2025

(30) Foreign Application Priority Data
May 16, 2023 (CN) 202310553454.0

(51) Int. Cl. A61B 5/11; A61B 5/00; A61B 5/05; A63B 23/20

(52) U.S. Cl. CPC A61B 5/1107; A61B 5/05; A61B 5/4561; A63B 23/20

(57) ABSTRACT

The present disclosure discloses a non-contact pelvic floor assessment and training method, belonging to the field of medical instruments. The present disclosure adopts a method of the non-contact pelvic floor exercise detection in combination with the magnetic stimulation to generate assessment templates for various indexes of the pelvic floor muscle, which can achieve active-passive combination assessment of the pelvic floor muscle; and at the same time, the present disclosure provides a non-contact pelvic floor training method, formulating a rehabilitation training program, and making a real-time adjustment to the training program based on changes in the assessment results of the pelvic floor muscle. In the aspect of the pelvic floor function assessment, the present disclosure can accurately reflect the functional state of its muscles and reduce the false positive rate diagnosed as the pelvic floor muscle dysfunction caused by the fact that the subject can not accurately perform the pelvic floor muscle contraction exercise; in the aspect of the training method, the present disclosure can assess the training quality in real-time during the training, adjust the training template in real-time, and guide the next rehabilitation training program based on the quality of the subject's current rehabilitation training, thereby achieving individualized and precise pelvic floor rehabilitation training.

### TECHNICAL FIELD

The present disclosure relates to the field of medical instruments, in particular to a non-contact pelvic floor assessment and training method.

### BACKGROUND OF THE INVENTION

Pelvic floor muscle diseases usually exhibit pelvic floor muscle contraction exercise problems, and pelvic floor dysfunction (PFD) is currently clinically manifested mainly as: pelvic organ prolapse (POP), Urinary Incontinence (UI), dyschezia, sexual dysfunction, fecal and urinary incontinence, pelvic floor chronic pain, and the like. Such diseases can cause anxiety, depression, low labor ability and social skills of a user, affect their psychological and physical health, and reduce the quality of life. According to related research reports, the prevalence rate of PFD also tends to rise year by year after the human beings enter an aging society.

The common assessment method for pelvic floor muscle diseases usually adopts a vaginal electrode to collect electromyographic signals inside the vagina for assessment, the electromyography measured by the method only has a certain correlation with the degree of the muscle contraction exercise within a certain range, but cannot directly reflect the degree of the muscle contraction, thereby it is difficult to accurately determine the functional condition of the pelvic floor muscle directly based on the magnitude of electromyographic values.

The current assessment method for the pelvic floor muscle is usually based on subjects performing an active contraction of the pelvic floor muscle according to a fixed template, and the pelvic floor muscle function of the subjects is assessed based on the parameters obtained during this process; some subjects that have not received a pelvic floor muscle exercise training are unable to accurately perform the pelvic floor muscle contraction exercise, leading to misdiagnosis as the pelvic floor muscle dysfunction; and, the results of active contraction of the pelvic floor muscle performed by subjects according to the template are greatly influenced by their subjective consciousness, which can lead to inaccurate assessment results easily; in addition, the current assessment of the pelvic floor muscle is mostly composed of parameters such as the maximum electromyography or pressure value and a rising time of a fast muscle, the average electromyography or pressure value of a slow muscle, and variability, which cannot determine the force exerting way, control ability, and nerve innervation function of the pelvic floor muscle of the subject, and the results are relatively one-sided.

The current method for training the pelvic floor muscle usually adopts a unified fixed template for training, a targeted individualized training template is not customized according to the assessment results, the training is not targeted, and it is impossible to adjust the training template in real-time based on the pelvic floor muscle exercise state of the subject during the training process, the rehabilitation training quality is poor, and the expected rehabilitation effect cannot be achieved.

### SUMMARY OF THE INVENTION

The purpose of the present disclosure is to provide a non-contact pelvic floor assessment and training method to solve the problems proposed in the background technology mentioned above.

In order to solve the above technical problems, the present disclosure provides the following technical solution: a non-contact pelvic floor assessment and training method.

According to the above technical solution, the assessment includes the following steps:
S1, determining whether the user's sitting posture and force exerting way during a magnetic stimulation are correct based on a non-contact biofeedback, thereby obtaining the correct sitting posture and the correct force exerting way on the pelvic floor muscle;
S2, obtaining a non-contact feedback waveform based on the user using correct sitting posture and correct force exerting way on the pelvic floor muscle, assessing the maximum contraction force of the pelvic floor muscle fast muscle and the maximum contraction force of the pelvic floor muscle slow muscle based on the non-contact feedback waveform respectively, and generating a pelvic floor muscle fast muscle assessment template and a pelvic floor muscle slow muscle assessment template respectively;
S3, assessing various functions of the pelvic floor muscle fast muscle and the pelvic floor muscle slow muscle according to the pelvic floor muscle fast muscle assessment template and the pelvic floor muscle slow muscle assessment template;
S4, assessing the user's neuromuscular regulation function by means of active-passive combination, obtaining a corresponding score based on various functional indexes of the pelvic floor muscle fast muscle, various functional indexes of the pelvic floor muscle slow muscle and a degree of neurological injury, thereby calculating an integrate score for the active-passive combination assessment of the pelvic floor muscle.

According to the above technical solution, in steps S1-S3, assessing various functions of the pelvic floor muscle fast muscle includes the following steps:
S2-1, collecting the non-contact feedback waveform for user to exert force using the pelvic floor muscle;
S2-2, setting a threshold grad* for the non-contact feedback waveform of the pelvic floor muscle according to the non-contact feedback database of the pelvic floor muscle, when the waveform is unimodal and the slope at the descent during relaxation is greater than grad*, it is determined that the user's sitting posture and force exerting way during the magnetic stimulation are correct; when the waveform is bimodal and the slope at the descent during relaxation is small, it is determined that the force exerting way is to use the anal sphincter muscle for contraction, and the sitting posture and force exerting way are incorrect;
determining the force exerting way by means of this waveform can effectively prevent the user from using an informal force exerting way that results in inaccurate assessment results, and when the user meets the correct sitting posture and the force exerting way is correct, the assessment will begin;
S2-3, using the maximum force to contract the pelvic floor muscle for x times rapidly when the user uses the correct sitting posture and the correct force exerting way on the pelvic floor muscle, thereby obtaining the non-contact feedback waveform;
typically, contracting the pelvic floor muscle for 5 times rapidly is optimal;
wherein, x is a constant set by a system;
S2-4, assessing the maximum contraction force S_{Fmax} of the pelvic floor muscle fast muscle: the user uses the correct sitting posture and the correct force exerting way on the pelvic floor muscle, and uses the maximum force to rapidly contract the pelvic floor muscle for multiple time, according to the amplitude set D={D₁, D₂, . . . , D_{N}} of the non-contact feedback waveform, wherein the maximum contraction amplitude corresponds to the maximum distance Dₘₐₓ=max (D) that the pelvic floor muscle of the subject can contract, the maximum contraction force S_{Fmax} of the user's pelvic floor muscle is obtained correspondingly, the pelvic floor muscle fast muscle assessment template is generated based on the fast muscle assessment template database corresponding to the actual value of S_{Fmax};
the collected parameters of the user's pelvic floor muscle contraction strength can be used to set the initial treatment intensity of magnetic stimulation, obtaining other muscle assessment parameters of the pelvic floor muscle of the subject, such as a control force of fast muscle, a fatigue level and an endurance of slow muscle, the personalized formulation of biofeedback training templates and so like;
S2-5, contracting the pelvic floor muscle for three times by user through using the correct sitting posture and the correct force exerting way on the pelvic floor muscle, the first pelvic floor muscle contraction force needs to reach a % of S_{Fmax}, the second pelvic floor muscle contraction force needs to reach b % of S_{Fmax}, and the third pelvic floor muscle contraction force needs to reach c % of S_{Fmax}, generating the fast muscle assessment template needing to contract the pelvic floor muscle for three times according to the fast muscle assessment template database corresponding to the actual value of the pelvic floor muscle contraction force;
the a, b and c are constants set by the system, and a<b<c;
S2-6, collecting the non-contact feedback waveform of the pelvic floor muscle contraction, and assessing a muscle strength

$${S}_{F}^{P}$$

of the user's pelvic floor muscle fast muscle according to the non-contact feedback waveform;
if S_{Fmax}<S_{std}, it indicates that the muscle strength

$${S}_{F}^{P}$$

of the user's fast muscle is weak, wherein S_{std} is a threshold for assessing the muscle strength of the fast muscle, which is set according to a database assessing big data;
S2-7, assessing a control force

$${S}_{F}^{C}$$

of the user's pelvic floor muscle fast muscle according to the non-contact feedback waveform, if a ratio N_{Fdown}/N_{F} of the number of data points N_{Fdown} in the lower portion of the template requirement line with the number of all data points N_{F} is >d %, it indicates that the user has poor control force

$${S}_{F}^{C}$$

on the fast muscle; if N_{Fdown}/N_{F}≤d %, it indicates that the user has normal control force

$${S}_{F}^{C}$$

on the fast muscle;
S2-8, assessing an explosive force

$${S}_{F}^{A}$$

of the user's pelvic floor muscle according to the non-contact feedback waveform: during a fast contraction-fast release, if

$${t}_{U P} \geq {t}_{UPstd}^{Ftop} ,$$

it indicates that the explosive force of the user's pelvic floor muscle is poor;

if

$${t}_{U P} < {t}_{UPstd}^{Ftop} ,$$

it indicates that the explosive force of the user's pelvic floor muscle is good;
wherein t_{UP} is a time taken to reach the peak of the amplitude, and

$${t}_{UPstd}^{Ftop}$$

is set as a time cut-off value taken from rest to the highest point reached by the contraction during a fast contraction-fast release;

S2-9, assessing the fatigue level

$${S}_{F}^{T}$$

of the user's fast muscle according to the non-contact feedback waveform, if after contracting the pelvic floor muscle for multiple times, the non-contact feedback waveform is manifested as: during a fast contraction-fast release, if

$${t}_{D O W N} \geq {t}_{DOWNstd}^{Ftop} ,$$

indicates that the user's pelvic floor muscle have entered into the fatigue period;
if

$${t}_{D O W N} < {t}_{DOWNstd}^{Ftop} ,$$

it indicates that the user's pelvic floor muscle have not yet entered into the fatigue period;
wherein t_{DOWN} is a time taken from the highest point to the lowest point of the amplitude in the waveform, and

$${t}_{D O WNstd}^{Ftop}$$

is a time cut-off value taken from the highest point reached by the contraction to rest during a fast contraction-fast release.

If the absolute value of the slope of the relaxation phase waveform is smaller than the absolute value of the slope of the template, it indicates that the user's relaxation process is too long, and the fast muscle has entered into the fatigue period, making it difficult to relax, which can be used to assess the fatigue level

$${S}_{F}^{T}$$

of the user's fast muscle and generate a subsequent personalized training correspondingly according to the fatigue level.

According to the above technical solution, in steps S1-S3, assessing various functions of the pelvic floor muscle slow muscle includes the following steps:
S3-1, contracting the pelvic floor muscle for three times by user through using the correct sitting posture and the correct force exerting way on the pelvic floor muscle, contracting the pelvic floor muscle to enable the feedback amplitude to reach e % of the maximum contraction force S_{Fmax} of the pelvic floor muscle fast muscle and maintain it for a period of time f, then continuing to contract the pelvic floor muscle without relaxing to enable the feedback waveform to reach the amplitude as high as possible and maintain it for a period of time g, and taking an average value of the feedback amplitudes in g to represent the maximum contraction force S_{Smax} of the user's pelvic floor muscle slow muscle;
as high as possible refers to the user tries his best to contract to a certain amplitude and maintain it for a period of time while performing a contraction maintenance;
as high as possible refers to the user tries his best to contract to a high amplitude and maintain it for a period of time, requiring the user to find a pattern that can have a high amplitude and maintain it for a long duration, it is a term in medicine that it often cannot be maintained for a long enough time when using the maximum force for contraction, therefore the user needs to find a force that can be maintained for a long time and have a high amplitude to contract;
the e, f, and g are constants set by the system;
S3-2, generating a pelvic floor muscle slow muscle assessment template based on the maximum contraction force S_{Smax} of the pelvic floor muscle slow muscle;
S3-3, taking h % of the maximum contraction force S_{Smax} amplitude of the pelvic floor muscle as the template amplitude for assessment, contracting and maintain the pelvic floor muscle by the user through using the correct sitting posture and the correct force exerting way on the pelvic floor muscle;
the h is a constant set by the system;
S3-4, assessing an endurance

$${S}_{S}^{E}$$

of the user's pelvic floor muscle slow muscle according to the non-contact feedback waveform of the pelvic floor muscle contraction, if the height specified by the template cannot be reached by the contraction maintenance or the time for the contraction maintenance cannot reach the length of the contraction time of the threshold, or the non-contact feedback waveform occurs to deviate from the template to trend to decrease during the contraction maintenance phase, it indicates that the endurance

$${S}_{S}^{E}$$

of the slow muscle is weak, on the contrary, it indicates that the endurance of the slow muscle is good;
S3-5, assessing the control force

$${S}_{S}^{C}$$

of the user's pelvic floor muscle slow muscle according to the non-contact feedback waveform of the pelvic floor muscle contraction: if the waveform occurs to shake during contraction and relaxation, it is manifested on the non-contact feedback waveform as: if the ratio N_{Sdown}/N of the number of data points N_{Sdown} in the lower portion of the template requirement line with the number of all data points N is >d %, it indicates the user has poor control force

$${S}_{S}^{C}$$

on the slow muscle, if N_{Sdown}/N≤d %, it indicates the user has good control force

$${S}_{S}^{C}$$

on the slow muscle.

It is manifested on the feedback waveform as: after a long period of contraction maintenance for the user's pelvic floor muscle, the slow muscle dominates in exerting force, when relaxing following the assessment template, different users have significant differences in the generated waveform due to different fatigue levels of the pelvic floor muscles, the pelvic floor muscle with lower fatigue level can quickly relax according to the template, while the pelvic floor muscle with higher fatigue level requires longer time for its muscle from tension to relaxation.

According to the above technical solution, in step S4, the active-passive combination assessment of the pelvic floor muscle includes the following steps:
S4-1, the user sits in the correct posture on the magnetic stimulation therapy device equipped with a biofeedback module, gradually increasing the magnetic stimulation intensity and obtaining passive contraction feedback of the pelvic floor muscle under the magnetic field stimulation;
S4-2, stimulating the user's pelvic floor muscle starting from a magnetic stimulation intensity of x₂, when the stimulation intensity reaches intensity S_{M}, the biofeedback amplitude of passive contraction of the pelvic floor muscle no longer increases with the increase of magnetic stimulation intensity, it indicates that the pelvic floor muscle have reached the passive contraction threshold after reaching the magnetic stimulation intensity S_{M};
wherein, x₂ is a constant set by the system;
S4-3, assessing the neurological injury of the pelvic floor muscle and its degree of injury according to the non-contact feedback waveform of passive contraction of the pelvic floor muscle: comparing the magnetic stimulation intensity S_{M} with the standard cut-off value

$${S}_{M}^{std}$$

to determine whether there is the neurological injury of the pelvic floor muscle and its degree of injury ε_{J};
wherein,

$${ε}_{J} = \{ \begin{matrix}{x}_{3} + ( {S}_{M} - {S}_{M}^{std} ) / {S}_{M} , {S}_{M} > {S}_{M}^{std} \\ {x}_{4} + ( {S}_{M} - {S}_{M}^{std} ) / {S}_{M} , {S}_{M} < {S}_{M}^{std}\end{matrix}$$

wherein, the standard cut-off value

$${S}_{M}^{std}$$

is a threshold which is generated based on a big data assessment database and is selected to distinguish the neurological injury of the pelvic floor muscle and its degree of injury;
S4-4, the calculation method for the integrate score M of the active-passive combination assessment of the pelvic floor muscle is:

$$M = \frac{α \cdot \frac{( {S}_{F}^{P} \cdot {S}_{F}^{C} \cdot {S}_{F}^{A} )}{{S}_{F}^{T}} + ( 1 - α ) \cdot \frac{{S}_{S}^{E} \cdot {S}_{S}^{C}}{{S}_{S}^{T}}}{{ε}_{J}}$$

wherein, α is a weight, and 0<α<1;

$${S}_{F}^{P}$$

indicates the muscle strength of the fast muscle,

$${S}_{F}^{C}$$

indicates the control force of the fast muscle,

$${S}_{F}^{A}$$

indicated the explosive force of the fast muscle,

$${S}_{F}^{T}$$

indicates the fatigue level of the fast muscle,

$${S}_{S}^{E}$$

indicates the fatigue level of the slow muscle,

$${S}_{S}^{C}$$

indicates the control force of the slow muscle,

$${S}_{S}^{T}$$

indicates the fatigue level of the slow muscle, ε_{J} indicates the degree of neurological injury.

According to the above technical solution, in step S4, the active-passive combination mode refers to:
the active mode is that the user contracts the pelvic floor muscle actively to obtain various indexes of the fast muscle and slow muscle of the pelvic floor muscle; the passive mode is the passive contraction feedback of the user's pelvic floor muscle under the magnetic field stimulation, to determine the existence of the user's pelvic floor muscle neurological injury and the degree of injury.

According to the above technical solution, the training method comprises the following steps:
S1, formulating a rehabilitation training program, which includes a neurological rehabilitation training program and a muscle rehabilitation training program;
S2, assessing pelvic floor muscle characteristic parameters during the rehabilitation training, and calculating a score for various functions of the pelvic floor muscle;
S3, making a real-time adjustment to the training program based on changes in the assessment results of the pelvic floor muscle.

According to the above technical solution, in step S1, the method of formulating a rehabilitation training program includes the following steps:
S7-1, integrating users whose magnetic stimulation intensity S_{M} in pelvic floor assessment is less than the corresponding magnetic stimulation intensity standard cut-off value

$${S}_{M}^{std}$$

into the neurological rehabilitation training, and formulating a passive auxiliary training with a total of k minutes for them, and there are m training templates in total, each training template has a duration of n, each training template contains p training actions;
the k, n, and p are constants set by the system;
the active training refers to the user performs two types of actions that are fast contraction-fast release and contraction maintenance actively according to the prompts on the display, the passive auxiliary training refers to the user performs the fast contraction-fast release action or the contraction maintenance action actively while applying the magnetic stimulation to assist and thereby to strengthen the actions;
S7-2, adjusting the proportion of fast contraction-fast release with contraction maintenance based on a score for various functions of the fast muscle and a score for various functions of the slow muscle, with the magnetic stimulation intensity being S_{M};
setting the standard cut-off values for the score for various functions of the fast muscle and the slow muscle according to the big data classification sample, and establishing the standard cut-off value set S_{FStd} for the score S_{F} for various functions of the fast muscle, the standard cut-off value set S_{FStd} includes the standard cut-off value

$${S}_{Fstd}^{P}$$

for the muscle strength of the fast muscle, the standard cut-off value

$${S}_{Fstd}^{C}$$

for the control force of the fast muscle, the standard cut-off value

$${s}_{Fstd}^{A}$$

for the explosive force of the fast muscle, and the standard cut-off value

$${S}_{Fstd}^{T}$$

for the fatigue level of the fast muscle, establishing the standard cut-off value set S_{Sstd} for the score Sₛ for various functions of the slow muscle, the standard cut-off value set S_{Sstd} includes the standard cut-off value

$${S}_{Fstd}^{E}$$

for the endurance of the slow muscle, the standard cut-off value

$${S}_{Fstd}^{C}$$

for the control force of the slow muscle and the standard cutoff value

$${S}_{Fstd}^{T}$$

for the fatigue level of the slow muscle;
if the score S_{F} for various functions of the fast muscle and the score S_{S} for various functions of the slow muscle are both lower than the corresponding standard cut-off values in the standard cut-off value sets S_{FStd}, S_{Sstd}, the corresponding fast contraction-fast release or contraction maintenance is allocated q; wherein, S_{F} includes

$${S}_{F}^{P} , {S}_{F}^{C} , {S}_{F}^{A} and {S}_{F}^{T} ,$$

Sₛ includes

$${S}_{S}^{E} , {S}_{S}^{C} and {S}_{S}^{T} ;$$

if only one of the score S_{F} for various functions of the fast muscle or the score S_{S} for various functions of the slow muscle is lower than the corresponding standard cut-off value in the corresponding standard cut-off value sets S_{Fstd}, S_{std}, the corresponding proportion of fast contraction-fast release action or contraction maintenance action increases by z % every time the score decreases by y, and the rest time between each action interval is at least r;

the q, y, z and r are constants set by the system;

S7-3, setting the cut-off value M_{std} for the active-passive combination assessment score of the pelvic floor muscle according to the big data classification sample, setting the standard cut-off value Esta for the degree of neurological injury, and obtaining the degree of neurological injury ε_{J}; if the degree of neurological injury ε_{J} is higher than ε_{std}, it indicates that the nerve is not injured; if the degree of neurological injury is lower than ε_{std}, it indicates the presence of neurological injury, and the smaller the value, the more severe the degree of injury; integrating the users whose degree of neurological injury ε_{J} in the pelvic floor assessment is higher than the standard cut-off value ε_{std} for the degree of neurological injury but whose active-passive combination assessment score M of the pelvic floor muscle is lower than its corresponding standard cut-off value M_{std} into the muscle rehabilitation training, formulating an active training program with a total duration of k₁ and m₁ training templates, wherein each training template has a duration of n₁ and each training template contains p₁ training actions;

the k₁, m₁, n₁ and p₁ are constants set by the system;

if the score S_{F} for various functions of the fast muscle and the score S_{S} for various functions of the slow muscle are both lower than the corresponding standard cut-off values in the standard cut-off value sets S_{FStd}, S_{Sstd} of the fast and slow muscle, the corresponding fast contraction-fast release or contraction maintenance are allocated q₁;
if only one of the score S_{F} for various functions of the fast muscle or the score S_{S} for various functions of the slow muscle is lower than the normal value, the corresponding proportion of fast contraction-fast release action or contraction maintenance action increases by z₁%, and the rest time between each action interval is at least r₁;

the q₁, z₁, and r₁ are constants set by the system.

According to the above technical solution, in steps S2-S3, the method of making a real-time adjustment to the training program includes the following steps:
S8-1, when the user engages in neurological rehabilitation training, calculating the score for various functions of the fast muscle, the score for various functions of the slow muscle, and active-passive combination assessment score of the pelvic floor muscle based on the passive contraction of the pelvic floor muscle during each magnetic stimulation auxiliary training; when the user engages in muscle rehabilitation training, calculating the score for various functions of the fast muscle, the score for various functions of the slow muscle, and active-passive combination assessment score of the pelvic floor muscle based on the training template in each fast contraction-fast release and contraction maintenance;
S8-2, when the user engages in neurological rehabilitation training:
if the active-passive combination assessment score of the pelvic floor muscle increases after the training of each training template is completed, the magnetic stimulation intensity decreases;
if the active-passive combination assessment score of the pelvic floor muscle decreases after the training of each training template is completed, the magnetic stimulation intensity increases;
if either score in

$${S}_{F}^{P} , {S}_{F}^{C} , {S}_{F}^{A} , {S}_{F}^{T} , {S}_{S}^{E} , {S}_{S}^{C} and {S}_{S}^{T}$$

decreases, the proportion of fast contraction-fast release or contraction maintenance increases correspondingly, and on the contrary, the corresponding proportion of fast contraction-fast release or contraction maintenance decreases;
if the scores of the fast muscle and the slow muscle decrease in the scores

$${S}_{F}^{P} , {S}_{F}^{C} , {S}_{F}^{A} , {S}_{F}^{T}$$

for various functions of the fast muscle and the scores

$${S}_{S}^{E} , {S}_{S}^{C} , {S}_{S}^{T}$$

for various functions of the slow muscle, the proportion remains unchanged;
S8-3, calculating the active-passive combination assessment score of the pelvic floor muscle after the training of each training template is completed, the magnetic stimulation intensity u decreases every time the score increases by s₁, when the score exceeds s₂, the magnetic stimulation intensity decreases to be u₀, and does not decrease any more; calculating various functions of the fast and slow muscles, when each function of the fast muscle or the slow muscle rises s₃, the corresponding proportion of fast contraction-fast release action or contraction maintenance action in one training template decreases by v %;
the s₁, u, s₂, u₀, s₃ and v are constants set by the system;
S8-4, when the user engages in muscle rehabilitation training:
if the active-passive combination assessment score of the pelvic floor muscle increases after the training of each training template is completed, the amplitude of the template increases;
if the active-passive combination assessment score of the pelvic floor muscle decreases after the training of each training template is completed, the amplitude of the template decreases;
if one of the score for various functions of the fast muscle or the score for various functions of the slow muscle decreases in the score for various functions of the fast muscle and the score for various functions of the slow muscle, the proportion of fast contraction-fast release or contraction maintenance increases correspondingly, on the contrary, the corresponding proportion decreases;
S8-5, calculating the active-passive combination assessment score of the pelvic floor muscle after each training template is completed, the template amplitude decreases by v₁% every time the score increases by s₄, when the score exceeds s₅, the template amplitude increases to the highest v₂% and does not increase any more; on the contrary, the template amplitude decreases to v₃% and does not decrease any more;
calculating the scores for various functions of the fast and slow muscles, when the score for various functions of the fast muscle or the score for various functions of the slow muscle increases by s₅, the corresponding proportion of fast contraction-fast release action or contraction maintenance action in one training template decreases by v₄%;
the s₄, v₁, s₅, v₂, v₃ and v₄ are constants set by the system.

Compared with the prior art, the present disclosure has the following beneficial effects: in the aspect of assessing the pelvic floor function, the present disclosure adopts the method of directly detecting the pelvic floor muscle exercise by electromagnetic waves, directly measures the state of the pelvic floor muscle exercise in real-time by the displacement, the speed, the acceleration and the dithering frequency and the like of the muscle exercise, can directly measure the parameters of the pelvic floor and accurately reflect the functional condition of the muscle based on this. Meanwhile, the active-passive combination assessment of the pelvic floor muscle can be realized by adopting a method of combining the non-contact pelvic floor exercise detection and the magnetic stimulation. The false positive rate of the disease diagnosed as the pelvic floor muscle dysfunction caused by the fact that the subject can not accurately perform the pelvic floor muscle contraction exercise can be reduced; in addition, the method can assess not only the conventional parameters of the pelvic floor muscle, but also the force exerting way, the control ability, the innervation function and the like of the pelvic floor muscle of the subject;
in the aspect of the treatment of pelvic floor dysfunction, the present disclosure formulates an individualized active-passive combination rehabilitation training program based on the pelvic floor muscle assessment characteristic parameters of the subject, in this program, the sequence and proportion of active training and passive training are adjusted according to the pelvic floor muscle assessment characteristic parameters mentioned above; the personalized active or passive training template is formulated according to the pelvic floor assessment characteristic parameters mentioned above, the training quality is assessed in real-time during the training process, and the training template of this training is adjusted in real-time accordingly; in addition, the next rehabilitation training program will be guided according to the quality of the subjects' current rehabilitation training, in order to achieve individualized and precise pelvic floor rehabilitation training.

### BRIEF DESCRIPTION OF THE DRAWINGS

The drawings are used to provide further understanding of the present disclosure and form a part of the specification, and the drawings are used to explain the present disclosure together with the embodiments of the present disclosure and do not constitute limitations on the present disclosure. In the drawings:

FIG. 1 is a schematic view of the active-passive combination assessment of the pelvic floor muscle in a non-contact pelvic floor assessment and training method of the present disclosure;

FIG. 2 is a schematic view of the fast muscle maximum contraction force assessment template in a non-contact pelvic floor assessment and training method of the present disclosure;

FIG. 3 is a schematic view of the fast muscle maximum muscle strength assessment in a non-contact pelvic floor evaluation and training method of the present disclosure;

FIG. 4 is a schematic view of the assessment for various functions of the fast muscle in a non-contact pelvic floor assessment and training method of the present disclosure;

FIG. 5 is a schematic view of the slow muscle maximum contraction force assessment template in a non-contact pelvic floor assessment and training method of the present disclosure;

FIG. 6 is a schematic view of the slow muscle maximum contraction force assessment 1 in a non-contact pelvic floor assessment and training method of the present disclosure;

FIG. 7 is a schematic view of the slow muscle maximum contraction force assessment 2 in a non-contact pelvic floor assessment and training method of the present disclosure;

FIG. 8 is a schematic view of the assessment template for various functions of the slow muscle in a non-contact pelvic floor assessment and training method of the present disclosure;

FIG. 9 is a schematic view of the assessment for various functions of the slow muscle in a non-contact pelvic floor assessment and training method of the present disclosure;

FIG. 10 is a schematic view of the assessment for various functions of the slow muscle in a non-contact pelvic floor assessment and training method of the present disclosure.

### DETAILED DESCRIPTION OF THE INVENTION

The technical solution in the embodiment of the present disclosure will be described clearly and comprehensively in conjunction with the drawings of the embodiment of the present disclosure below. It is evident that the described embodiment is merely a part of the embodiments of the present disclosure, rather than all embodiments. Based on the embodiments of the present disclosure, all other embodiments obtained by ordinary technicians in the field without creative efforts fall within the scope of protection of the present disclosure.

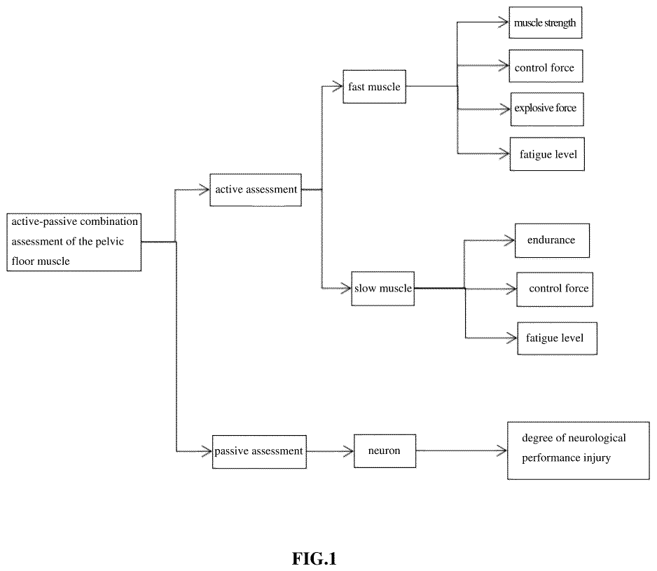

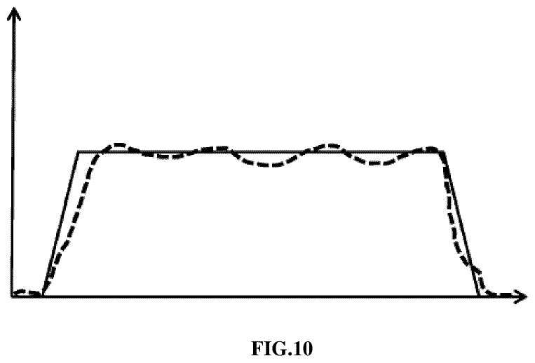

Referring to FIG. 1-10, the technical solution provided by the present disclosure is as follows:

A non-contact pelvic floor assessment, the assessment includes the following steps:
S1, determining whether the user's sitting posture and force exerting way during a magnetic stimulation are correct based on a non-contact biofeedback, thereby obtaining the correct sitting posture and the correct force exerting way on the pelvic floor muscle;
S2, obtaining a non-contact feedback waveform based on the user's correct sitting posture and correct force exerting way on the pelvic floor muscle, assessing the maximum contraction force of the pelvic floor muscle fast muscle and the maximum contraction force of the pelvic floor muscle slow muscle based on the non-contact feedback waveform respectively, and generating a pelvic floor muscle fast muscle assessment template and a pelvic floor muscle slow muscle assessment template respectively;
S3, assessing various functions for the pelvic floor muscle fast muscle and the pelvic floor muscle slow muscle according to the pelvic floor muscle fast muscle assessment template and the pelvic floor muscle slow muscle assessment template;
S4, assessing the user's neuromuscular regulation function by means of active-passive combination, obtaining a corresponding score based on various functional indexes of the pelvic floor muscle fast muscle, various functional indexes of the pelvic floor muscle slow muscle and a degree of neurological injury, thereby calculating an integrate score for the active-passive combination assessment of the pelvic floor muscle.

In steps S1-S3, assessing various functions of the pelvic floor muscle fast muscle includes the following steps:
S2-1, collecting the non-contact feedback waveform for user to exert force using the pelvic floor muscle;
S2-2, setting a threshold grad* for the non-contact feedback waveform of the pelvic floor muscle according to the non-contact feedback database of the pelvic floor muscle, when the waveform is unimodal and the slope at the descent during relaxation is greater than grad*, it is determined that the user's sitting posture and force exerting way during the magnetic stimulation are correct; when the waveform is bimodal and the slope at the descent during relaxation is small, it is determined that the force exerting way is to use the anal sphincter muscle for contraction, and the sitting posture and force exerting way are incorrect;
S2-3, using the maximum force to contract the pelvic floor muscle for x times rapidly when the user uses the correct sitting posture and the correct force exerting way on the pelvic floor muscle, thereby obtaining the non-contact feedback waveform;
wherein, x is a constant set by a system;
S2-4, assessing the maximum contraction force S_{Fmax} of the pelvic floor muscle fast muscle: the user uses the correct sitting posture and the correct force exerting way on the pelvic floor muscle, and uses the maximum force to rapidly contract the pelvic floor muscle for multiple time, according to the amplitude set D={D₁, D₂, . . . , D_{N}} of the non-contact feedback waveform, wherein the maximum contraction amplitude corresponds to the maximum distance Dₘₐₓ=max(D) that the pelvic floor muscle of the subject can contract, the maximum contraction force S_{Fmax} of the user's pelvic floor muscle is obtained correspondingly, the pelvic floor muscle fast muscle assessment template is generated based on the fast muscle assessment template database corresponding to the actual value of S_{Fmax};
S2-5, contracting the pelvic floor muscle for three times by user through using the correct sitting posture and the correct force exerting way on the pelvic floor muscle, the first pelvic floor muscle contraction force needs to reach a % of S_{Fmax}, the second pelvic floor muscle contraction force needs to reach b % of S_{Fmax}, and the third pelvic floor muscle contraction force needs to reach c % of S_{Fmax}, generating the fast muscle assessment template needing to contract the pelvic floor muscle for three times according to the fast muscle assessment template database corresponding to the actual value of the pelvic floor muscle contraction force;
the a, b and c are constants set by the system, and a<b<c;
S2-6, collecting the non-contact feedback waveform of the pelvic floor muscle contraction, and assessing a muscle strength

$${S}_{F}^{P}$$

of the user's pelvic floor muscle fast muscle according to the non-contact feedback waveform;
if S_{Fmax}<S_{std}, it indicates that the muscle strength

$${S}_{F}^{P}$$

of the user's fast muscle is weak, wherein S_{std} is a threshold for assessing the muscle strength of the fast muscle, which is set according to a database assessing big data;
S2-7, assessing a control force

$${S}_{F}^{C}$$

of the user's pelvic floor muscle fast muscle according to the non-contact feedback waveform, if a ratio N_{Fdown}/N_{F} of the number of data points N_{Fdown} in the lower portion of the template requirement line with the number of all data points N_{F} is >d %, it indicates that the user has poor control force

$${S}_{F}^{C}$$

on the fast muscle; if N_{Fdown}/N_{F}≤d %, it indicates that the user has normal control force

$${S}_{F}^{C}$$

on the fast muscle;
S2-8, assessing an explosive force

$${S}_{F}^{A}$$

of the user's pelvic floor muscle according to the non-contact feedback waveform: during a fast contraction-fast release, if

$${t}_{UP} \geq {t}_{UPstd}^{Ftop} ,$$

it indicates that the explosive force of the user's pelvic floor muscle is poor;
if

$${t}_{UP} < {t}_{UPstd}^{Ftop} ,$$

it indicates that the explosive force of the user's pelvic floor muscle is good;
wherein t_{UP} is a time taken to reach the peak of the amplitude, and

$${t}_{UPstd}^{Ftop}$$

is a time cut-off value taken from rest to the highest point reached by the contraction during a fast contraction-fast release;
S2-9, assessing the fatigue level

$${S}_{F}^{T}$$

of the user's fast muscle according to the non-contact feedback waveform, if after contracting the pelvic floor muscle for multiple times, the non-contact feedback waveform is manifested as: during a fast contraction-fast release, if

$${t}_{DOWN} \geq {t}_{DOWNstd}^{Ftop} ,$$

it indicates that the user's pelvic floor muscle have entered into the fatigue period;
if

$${t}_{D O W N} < {t}_{DOWNstd}^{Ftop} ,$$

it indicates that the user's pelvic floor muscle have not yet entered into the fatigue period;
wherein t_{DOWN} is a time taken from the highest point to the lowest point of the amplitude in the waveform, and

$${t}_{DOWNstd}^{Ftop}$$

is a time cut-off value taken from the highest point reached by the contraction to rest during a fast contraction-fast release.

In steps S1-S3, assessing various functions of the pelvic floor muscle slow muscle includes the following steps:
S3-1, contracting the pelvic floor muscle for three times by user through using the correct sitting posture and the correct force exerting way on the pelvic floor muscle, contracting the pelvic floor muscle to enable the feedback amplitude to reach e % of the maximum contraction force S_{Fmax} of the pelvic floor muscle fast muscle and maintain it for a period of time f, then continuing to contract the pelvic floor muscle without relaxing to enable the feedback waveform to reach the amplitude as high as possible and maintain it for a period of time g, and taking an average value of the feedback amplitudes in g to represent the maximum contraction force S_{Smax} of the user's pelvic floor muscle slow muscle;
as high as possible refers to the user tries his best to contract to a certain amplitude and maintain it for a period of time while performing a contraction and maintenance;
the e, f, and g are constants set by the system;
S3-2, generating a pelvic floor muscle slow muscle assessment template based on the maximum contraction force S_{Smax} of the pelvic floor muscle slow muscle;
S3-3, taking h % of the maximum contraction force S_{Smax} amplitude of the pelvic floor muscle as the template amplitude for assessment, contracting and maintain the pelvic floor muscle by the user through using the correct sitting posture and the correct force exerting way on the pelvic floor muscle;
the h is a constant set by the system;
S3-4, assessing an endurance

$${S}_{S}^{E}$$

of the user's pelvic floor muscle slow muscle according to the non-contact feedback waveform of the pelvic floor muscle contraction, if the height specified by the template cannot be reached by the contraction maintenance or the time for the contraction maintenance cannot reach the length of the contraction time of the threshold, or the non-contact feedback waveform occurs to deviate from the template to trend to decrease during the contraction maintenance phase, it indicates that the endurance

$${S}_{S}^{E}$$

of the slow muscle is weak, on the contrary, it indicates that the endurance of the slow muscle is good;
S3-5, assessing the control force

$${S}_{S}^{C}$$

of the user's pelvic floor muscle slow muscle according to the non-contact feedback waveform of the pelvic floor muscle contraction: if the waveform occurs to shake during contraction and relaxation, it is manifested on the non-contact feedback waveform as: if the ratio N_{Sdown}/N of the number of data points N_{Sdown} in the lower portion of the template requirement line with the number of all data points N is >d %, it indicates the user has poor control force

$${S}_{S}^{C}$$

on the slow muscle, if N_{Sdown}/N≤d %, it indicates the user has good control force

$${S}_{S}^{C}$$

on the slow muscle.

In step S4, the active-passive combination assessment of the pelvic floor muscle includes the following steps:
S4-1, the user sits in the correct posture on the magnetic stimulation therapy device equipped with a biofeedback module, gradually increasing the magnetic stimulation intensity and obtaining passive contraction feedback of the pelvic floor muscle under the magnetic field stimulation;
S4-2, stimulating the user's pelvic floor muscle starting from a magnetic stimulation intensity of x₂, when the stimulation intensity reaches S_{M}, the biofeedback amplitude of passive contraction of the pelvic floor muscle no longer increases with the increase of magnetic stimulation intensity, it indicates that the pelvic floor muscle have reached the passive contraction threshold after reaching the magnetic stimulation intensity S_{M};
wherein, x₂ is a constant set by the system;
S4-3, assessing the neurological injury of the pelvic floor muscle and its degree of injury according to the non-contact feedback waveform of passive contraction of the pelvic floor muscle: comparing the magnetic stimulation intensity S_{M} with the standard cut-off value

$${S}_{M}^{std}$$

to determine whether there is the neurological injury of the pelvic floor muscle and its degree of injury ε_{J};
wherein,

$${ε}_{J} = \{ \begin{matrix}{x}_{3} + ( {S}_{M} - {S}_{M}^{std} ) / {S}_{M} , {S}_{M} > {S}_{M}^{std} \\ {x}_{4} + ( {S}_{M} - {S}_{M}^{std} ) / {S}_{M} , {S}_{M} < {S}_{M}^{std}\end{matrix}$$

wherein, the standard cut-off value

$${S}_{M}^{std}$$

is a threshold which is generated based on a big data assessment database and is selected to distinguish the neurological injury of the pelvic floor muscle and its degree of injury;
S4-4, the calculation method for the integrate score M of the active-passive combination assessment of the pelvic floor muscle is:

$$M = \frac{α \cdot \frac{( {S}_{F}^{P} \cdot {S}_{F}^{C} \cdot {S}_{F}^{A} )}{{S}_{F}^{T}} + ( 1 - α ) \cdot \frac{{S}_{S}^{E} \cdot {S}_{S}^{C}}{{S}_{S}^{T}}}{{ε}_{I}}$$

wherein, α is a weight, and 0<α<1;

$${S}_{F}^{P}$$

indicates the muscle strength of the fast muscle,

$${S}_{F}^{C}$$

indicates the control force of the fast muscle,

$${S}_{F}^{A}$$

indicates the explosive force of the fast muscle,

$${S}_{F}^{T}$$

indicates the fatigue level of the fast muscle,

$${S}_{S}^{E}$$

indicates the endurance of the slow muscle,

$${S}_{S}^{C}$$

indicates the control force of the slow muscle,

$${S}_{S}^{T}$$

indicates the fatigue level of the slow muscle, ε_{J} indicates the degree of neurological injury.

In step S4, the active-passive combination mode refers to:
the active mode is that the user contracts the pelvic floor muscle actively to obtain various indexes of the fast muscle and slow muscle of the pelvic floor muscle; the passive mode is the passive contraction feedback of the user's pelvic floor muscle under the magnetic field stimulation, to determine the existence of the user's pelvic floor muscle neurological injury and the degree of injury.

A non-contact pelvic floor training method, the training method comprises the following steps:
S1, formulating a rehabilitation training program, which includes a neurological rehabilitation training program and a muscle rehabilitation training program;
S2, assessing pelvic floor muscle characteristic parameters during the rehabilitation training, and calculating a score for various functions of the pelvic floor muscle;
S3, making a real-time adjustment to the training program based on changes in the assessment results of the pelvic floor muscle.

In step S1, the method of formulating a rehabilitation training program includes the following steps:
S7-1, integrating users whose magnetic stimulation intensity S_{M} in pelvic floor assessment is less than the corresponding magnetic stimulation intensity standard cut-off value

$${S}_{M}^{std}$$

into the neurological rehabilitation training, and formulating a passive auxiliary training with a total of k minutes for them, there are m training templates in total, each training template has a duration of n, each training template contains p training actions;
the k, n, and p are constants set by the system;
the active training refers to the user performs two types of actions that are fast contraction-fast release and contraction maintenance actively according to the prompts on the display, the passive auxiliary training refers to the user performs the fast contraction-fast release or contraction maintenance action actively while applying the magnetic stimulation to assist and thereby to strengthen the actions;
S7-2, adjusting the proportion of fast contraction-fast release with contraction maintenance based on a score for various functions of the fast muscle and a score for various functions of the slow muscle, with the magnetic stimulation intensity being S_{M};
setting the standard cut-off values for the score for various functions of the fast muscle and the slow muscle according to the big data classification sample, and establishing the standard cut-off value set S_{Fstd} for the score S_{F} for various functions of the fast muscle, the standard cut-off value set S_{FStd} includes the standard cut-off value

$${S}_{Fstd}^{P}$$

for the muscle strength of the fast muscle, the standard cut-off value

$${S}_{Fstd}^{C}$$

for the control force of the fast muscle, the standard cut-off value

$${S}_{Fstd}^{A}$$

for the explosive force of the fast muscle, and the standard cut-off value

$${S}_{Fstd}^{T}$$

for the fatigue level of the fast muscle, establishing the standard cut-off value set S_{Sstd} for the score Sₛ for various functions of the slow muscle, the standard cut-off value set S_{Sstd} includes the standard cut-off value

$${S}_{Fstd}^{E}$$

for the endurance of the slow muscle, the standard cut-off value

$${S}_{Fstd}^{C}$$

for the control force of the slow muscle and the standard cutoff value

$${S}_{Fstd}^{T}$$

for the fatigue level of the slow muscle;
if the score S_{F} for various functions of the fast muscle and the score S_{S} for various functions of the slow muscle are both lower than the corresponding standard cut-off values in the standard cut-off value sets S_{FStd}, S_{Sstd}, the corresponding fast contraction-fast release or contraction maintenance is allocated q; wherein, S_{F} includes

$${S}_{F}^{P} , {S}_{F}^{C} , {S}_{F}^{A} and {S}_{F}^{T} ;$$

Sₛ includes

$${S}_{S}^{E} , {S}_{S}^{C} and {S}_{S}^{T} ;$$

if only one of the score S_{F} for various functions of the fast muscle or the score S_{S} for various functions of the slow muscle is lower than the corresponding standard cut-off value in the corresponding standard cut-off value sets S_{Fstd}, S_{std}, the corresponding proportion of fast contraction-fast release or contraction maintenance actions increases by z % every time the score decreases by y, and the rest time between each action interval is at least r;
the q, y, z and r are constants set by the system;
S7-3, setting the cut-off value M_{std} for the active-passive combination assessment score of the pelvic floor muscle according to the big data classification sample, setting the standard cut-off value ε_{std} for the degree of neurological injury, and obtaining the degree of neurological injury ε_{J}; if the degree of neurological injury ε_{J}, is higher than ε_{std}, it indicates that the nerve is not injured; if the degree of neurological injury is lower than ε_{std}, it indicates the presence of neurological injury, and the smaller the value, the more severe the degree of injury; integrating the users whose degree of neurological injury ε_{J} in the pelvic floor assessment is higher than the standard cut-off value ε_{std} for the degree of neurological injury but whose active-passive combination assessment score M of the pelvic floor muscle is lower than its corresponding standard cut-off value M_{std} into the muscle rehabilitation training, formulating an active training program with a total duration of k₁ and m₁ training templates, wherein each training template has a duration of n₁ and each training template contains p₁ training actions;
the k₁, m₁, n₁ and p₁ are constants set by the system;
if the score S_{F} for various functions of the fast muscle and the score S_{S} for various functions of the slow muscle are both lower than the corresponding standard cut-off values in the standard cut-off value sets S_{FStd}, S_{Sstd} of the fast and slow muscle, the corresponding fast contraction-fast release or contraction maintenance actions are allocated q₁;

if only one of the score S_{F} for various functions of the fast muscle or the score S_{S} for various functions of the slow muscle is lower than the normal value, the corresponding proportion of fast contraction-fast release action or contraction maintenance action increases by z₁%, and the rest time between each action interval is at least r₁;
the q₁, z₁, and r₁ are constants set by the system.

In steps S2-S3, the method of making a real-time adjustment to the training program includes the following steps:
S8-1, when the user engages in neurological rehabilitation training, calculating the score for various functions of the fast muscle, the score for various functions of the slow muscle, and active-passive combination assessment score of the pelvic floor muscle based on the passive contraction of the pelvic floor muscle during each magnetic stimulation auxiliary training; when the user engages in muscle rehabilitation training, calculating the score for various functions of the fast muscle, the score for various functions of the slow muscle, and active-passive combination assessment score of the pelvic floor muscle based on the training template in each fast contraction-fast release and contraction maintenance;
S8-2, when the user engages in neurological rehabilitation training:
if the active-passive combination assessment score of the pelvic floor muscle increases after the training of each training template is completed, the magnetic stimulation intensity decreases;
if the active-passive combination assessment score of the pelvic floor muscle decreases after the training of each training template is completed, the magnetic stimulation intensity increases;
if either score in

$${S}_{F}^{P} , {S}_{F}^{C} , {S}_{F}^{A} , {S}_{F}^{T} , {S}_{S}^{E} , {S}_{S}^{C} and {S}_{S}^{T}$$

decreases, the proportion of fast contraction-fast release or contraction maintenance increases correspondingly, and on the contrary, the corresponding proportion of fast contraction-fast release or contraction maintenance decreases; if the scores of the fast muscle and the slow muscle decrease in the scores

$${S}_{F}^{P} , {S}_{F}^{C} , {S}_{F}^{A} , {S}_{F}^{T}$$

for various functions of the fast muscle and the scores

$${S}_{S}^{E} , {S}_{S}^{C} , {S}_{S}^{T}$$

for various functions of the slow muscle, the proportion remains unchanged;
S8-3, calculating the active-passive combination assessment score of the pelvic floor muscle after the training of each training template is completed, the magnetic stimulation intensity u decreases every time the score increases by s₁, when the score exceeds s₂, the magnetic stimulation intensity decreases to be u₀, and does not decrease any more; calculating various functions of the fast and slow muscles, when each function of the fast muscle or the slow muscle rises s₃, the corresponding proportion of fast contraction-fast release action or contraction maintenance action in one training template decreases by v %;
the s₁, u, s₂, u₀, s₃ and v are constants set by the system;
S8-4, when the user engages in muscle rehabilitation training:
if the active-passive combination assessment score of the pelvic floor muscle increases after the training of each training template is completed, the amplitude of the template increases;
if the active-passive combination assessment score of the pelvic floor muscle decreases after the training of each training template is completed, the amplitude of the template decreases;
if one of the score for various functions of the fast muscle or the score for various functions of the slow muscle decreases in the score for various functions of the fast muscle and the score for various functions of the slow muscle, the proportion of fast contraction-fast release or contraction maintenance increases correspondingly, on the contrary, the corresponding proportion decrease;
S8-5, calculating the active-passive combination assessment score of the pelvic floor muscle after each training template is completed, the template amplitude decreases by v₁% every time the score increases by s₄, when the score exceeds s₅, the template amplitude increases to the highest v₂% and does not increase any more; on the contrary, the template amplitude decreases to v₃% and does not decrease any more;
calculating the scores for various functions of the fast and slow muscles, when the score for various functions of the fast muscle or the score for various functions of the slow muscle increases by s₅, the corresponding proportion of fast contraction-fast release action or contraction maintenance action in one training template decreases by v₄%;
the s₄, v₁, s₅, v₂, v₃ and v₄ are constants set by the system.

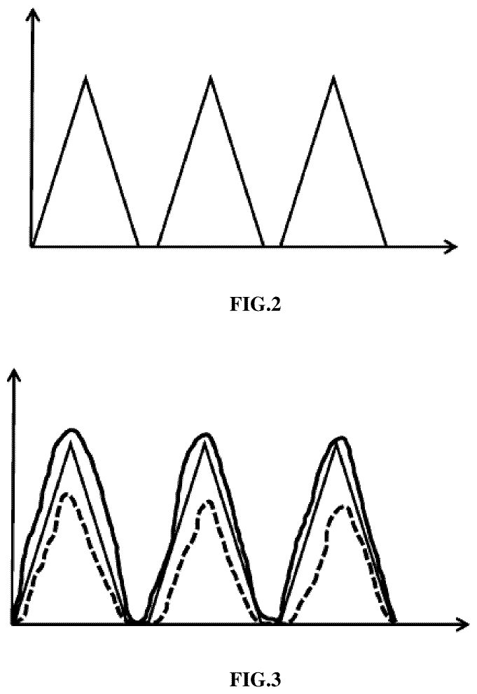

In this embodiment:
determining whether the user's sitting posture and force exerting way during a magnetic stimulation are correct based on a non-contact biofeedback, thereby obtaining the correct sitting posture and the correct force exerting way on the pelvic floor muscle, when the pelvic floor muscle is used usually for contraction, the non-contact feedback waveform is unimodal and has a larger slope at the descent during relaxation, if the anal sphincter muscle is used for contraction, a bimodal wave will appear and the slope at the descent during relaxation is small. As shown in FIG. 2, the feedback waveform obtained from the user's correct sitting posture and correct force exerting way on the pelvic floor muscle is represented;
when the user meets the correct sitting posture and the force exerting way is correct, assessing various functions of the pelvic floor muscle fast muscle according to the assessment template shown in FIG. 3: the user uses the maximum force to rapidly contract the pelvic floor muscle for multiple time, obtaining the amplitude set D={D₁, D₂, . . . , D_{N}} of the waveform based on feedback, wherein the maximum contraction amplitude corresponds to the maximum distance Dₘₐₓ=max(D) that the pelvic floor muscle of the subject can contract, that is, the user's pelvic floor muscle contraction maximum intensity Sₘₐₓ. Wherein, the solid line represents the assessment template, and the solid curve and the dashed curve represent two different types of contraction feedback waveforms.

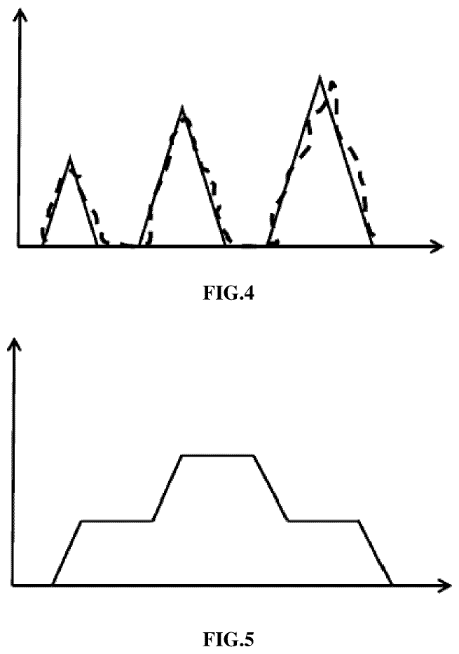

Generating the assessment template needing to contract the pelvic floor muscle for three times, wherein, the first pelvic floor muscle contraction force needs to reach 60% of S_{Fmax}, the second pelvic floor muscle contraction force needs to reach 80% of S_{Fmax}, and the third pelvic floor muscle contraction force needs to reach 100% of S_{Fmax}, as shown in FIG. 4;
assessing a muscle strength

$${S}_{F}^{P}$$

of the user's pelvic floor muscle fast muscle according to the non-contact feedback waveform: if the maximum contraction force S_{Fmax} of the pelvic floor muscle is smaller than the standard cut-off value S_{Sstd}, that is S_{Fmax}<S_{std}, it indicates that the muscle strength

$${S}_{F}^{P}$$

of the user's fast muscle is weak;
assessing a control force

$${S}_{F}^{C}$$

of the user's pelvic floor muscle fast muscle according to the non-contact feedback waveform, if the waveform occurs to shake during contraction and relaxation, it is manifested in the feedback waveform as: a ratio of the number of data points N_{Fdown} in the lower portion of the template requirement line with the number of all data points N_{F} is greater than 30%, that is N_{Fdown}/N_{F}>30%, it indicates that the user has poor control force

$${S}_{F}^{C}$$

on the fast muscle;
assessing an explosive force

$${S}_{F}^{A}$$

of the user's pelvic floor muscle according to the non-contact feedback waveform, if the slope of the waveform during contraction and relaxation is large, it indicates that the user's pelvic floor muscle contracts at a faster rate, corresponding to the explosive force

$${S}_{F}^{A}$$

of the user's pelvic floor muscle is good, on the contrary, it indicates that the user's pelvic floor muscle contracts at a slower rate, corresponding to the explosive force

$${S}_{F}^{A}$$

of the user's pelvic floor muscle is poor; it is manifested in the feedback waveform as: during a fast contraction-fast release, if

$${t}_{U P} \geq {t}_{UPstd}^{Ftop} ,$$

it indicates that the explosive force of the user's pelvic floor muscle is poor;
if

$$if {t}_{U P} < {t}_{UPstd}^{Ftop} ,$$

it indicated that the explosive force of the user's pelvic floor muscle is good;
wherein t_{UP} is a time taken to reach the peak of the amplitude,

$${t}_{UPstd}^{Ftop}$$

is a time cut-off value taken from rest to the highest point reached by the contraction during a fast contraction-fast release;
assessing the fatigue level

$${S}_{F}^{T}$$

of the user's fast muscle according to the non-contact feedback waveform: if, the waveform of the fast muscle during the relaxation phase of the fast contraction-fast release action occurs to a small slope after multiple assessment, it is manifested in the waveform as: during a fast contraction-fast release, if

$${t}_{D O W N} \geq {t}_{DOWNstd}^{Ftop} ,$$

it indicated that the user's pelvic floor muscle have entered into the fatigue period;
if

$$if {t}_{D O W N} < {t}_{DOWNstd}^{Ftop} ,$$

it indicates that the user's pelvic floor muscle have not yet entered into the fatigue period;
wherein t_{DOWN} is a time taken from the highest point to the lowest point of the amplitude in the waveform,

$${t}_{DOWNstd}^{Ftop}$$

is a time cut-off value taken from the highest point reached by the contraction to rest during a fast contraction-fast release.

As shown in FIG. 4, the folded line represents the assessment template, and the dashed line represents the feedback waveform of fast contraction-fast release of the fast muscle during the assessment: that is if the absolute value of the slope of waveform in the relaxation phase is smaller than the absolute value of the template slope, it indicates that the user's relaxation process is too long and the fast muscle has entered a fatigue period, making it difficult to relax, which can be used to assess the fatigue level

$${S}_{F}^{T}$$

of the user's fast muscle and generate a subsequent personalized training correspondingly according to the fatigue level.

When the user meets the correct sitting posture and the force exerting way is correct, assessing various functions of the pelvic floor muscle slow muscle: assessing the maximum contraction force S_{Smax} of the pelvic floor muscle slow muscle: after mastering how to correctly exert force on the pelvic floor muscle in the correct posture, the user can assess the maximum contraction force of the pelvic floor muscle slow muscle. The user assesses the maximum contraction force of the pelvic floor muscle slow muscle according to the template shown in FIG. 4 below, firstly, the patient needs to contract the pelvic floor muscle to enable a feedback amplitude to reach 45% of the maximum contraction force S_{Fmax} of the pelvic floor muscle fast muscle and maintain it for 3 seconds, because muscle engagement is highest in the slow muscle after the muscle contraction is maintain for 3 seconds; then continues to contract the pelvic floor muscle without relaxing to enable the feedback amplitude to reach the amplitude as high as possible and maintain it for 10 seconds, and taking an average value of the feedback amplitudes in 10 seconds to represent the maximum contraction force S_{Smax} of the user's pelvic floor muscle slow muscle, as shown in FIG. 5, the slow muscle maximum contraction force assessment template.

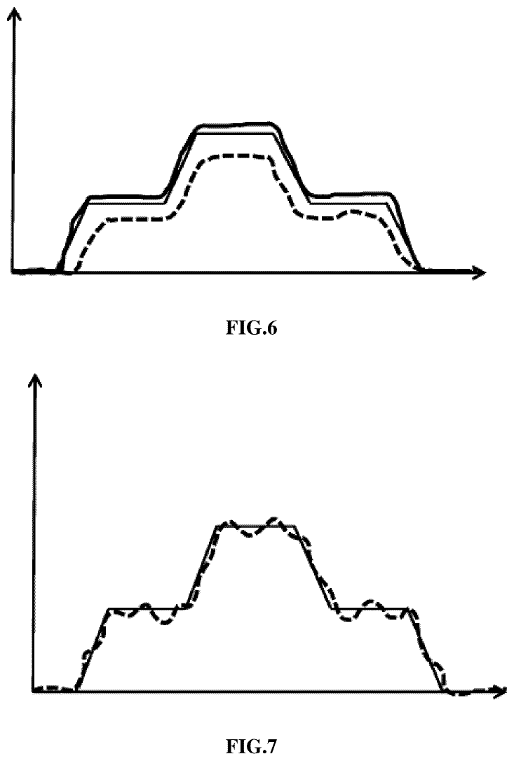

As shown in FIG. 6-10, the folder line diagram is a template diagram of pelvic floor muscle contraction, wherein, the solid line represents the template that requires the user to contract according to the line, and the solid curve and dashed curve represent two different situations of non-contact feedback waveforms obtained by the user's actual contraction of the pelvic floor muscle; furthermore, the solid curve represents the non-contact feedback waveform of the pelvic floor muscle above the template, the dashed curve represents the non-contact feedback waveform of the pelvic floor muscle below the template.
assessing an endurance

$${S}_{S}^{E}$$

of the user's pelvic floor muscle slow muscle according to the non-contact feedback waveform of the pelvic floor muscle contraction: as shown in FIG. 6, 75% of the maximum contraction force S_{Smax} of the pelvic floor muscle is taken as the template amplitude for assessment, the user is required to contract and maintain the pelvic floor muscle according to the template and the correct force exerting way in the correct posture, the various functions of the pelvic floor muscle can be assessed based on the feedback results, the endurance

$${S}_{S}^{E}$$

of the user's slow muscle can be assessed based on the length of contraction time reaching the threshold.

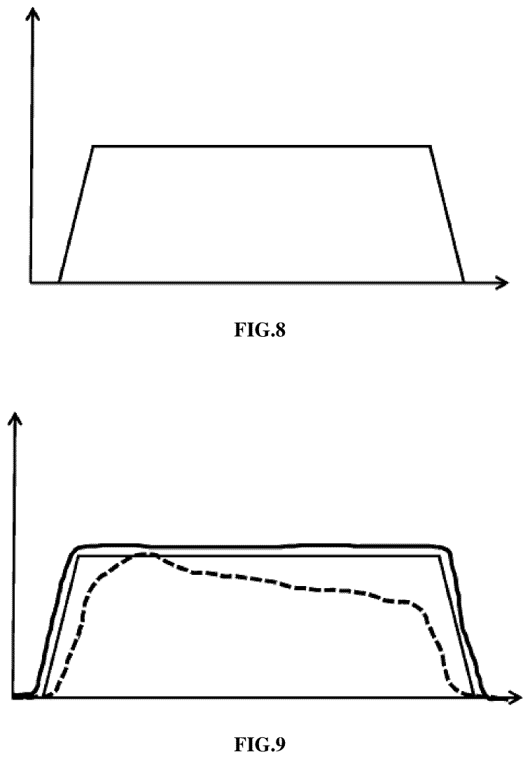

As shown by the dashed curves in FIGS. 7, 9 and 10: the user contracts the pelvic floor muscle but the height specified by the template cannot be maintained stability and reached, or the time for the contraction maintenance cannot reach the time required by the template, the feedback results of the waveform occurs to deviate from the template to trend to decrease during the contraction maintenance phase, it indicates that the endurance

$${S}_{S}^{E}$$

of the user's slow muscle is weak;
assessing an endurance

$${S}_{S}^{E}$$

of the user's pelvic floor muscle slow muscle according to the non-contact feedback waveform of the pelvic floor muscle contraction: if the waveform occurs to shake during contraction and relaxation, it is manifested on the feedback waveform as: if the ratio of the number of data points N_{Sdown} in the lower portion of the template requirement line with the number of all data points N is greater than 30%, that is N_{Sdown}/N>30%, it indicates the user has poor control force

$${S}_{S}^{C}$$

on the slow muscle, N_{Sdown}/N≤30%, it indicates the user has good control force

$$force {S}_{S}^{C}$$

on the slow muscle;
assessing the fatigue level

$${S}_{F}^{T}$$

of the user's pelvic floor muscle slow muscle according to the relaxation phase of contraction maintenance: it is manifested on the feedback waveform as, after a long period of contraction maintenance for the user's pelvic floor muscle, the slow muscle dominates in exerting force, when relaxing following the assessment template, different users have significant differences in the generated waveform due to different fatigue levels of the pelvic floor muscles, the pelvic floor muscle with lower fatigue level can quickly relax the pelvic floor muscle according to the template, while the pelvic floor muscle with higher fatigue level requires longer time for its muscle from tension to relaxation;
calculating the assessment score M by means of the active-passive combination assessment of the pelvic floor muscle: the user sits in the correct posture on the magnetic stimulation therapy device equipped with a biofeedback module, gradually increasing the magnetic stimulation intensity and obtaining passive contraction feedback of the pelvic floor muscle under the magnetic field stimulation; stimulating the user's pelvic floor muscle starting from a magnetic stimulation intensity of 1, as the magnetic stimulation intensity gradually increases, the biofeedback amplitude of passive contraction of the pelvic floor muscle gradually increases, however, when the stimulation intensity reaches an intensity S_{M}, the biofeedback amplitude of passive contraction of the pelvic floor muscle does not increase any more with the increase of magnetic stimulation intensity, it indicates that the pelvic floor muscle have reached the passive contraction threshold after reaching the magnetic stimulation intensity S_{M}, comparing the magnetic stimulation intensity with the standard cut-off value to determine whether there is the neurological injury of the pelvic floor muscle and its degree of injury ε_{J};
the integrate score M of the active-passive combination assessment of the pelvic floor muscle proposed by the present disclosure is obtained by the following parameters:

$$M = \frac{α \cdot \frac{( {S}_{F}^{P} \cdot {S}_{F}^{C} \cdot {S}_{F}^{A} )}{{S}_{F}^{T}} + ( 1 - α ) \cdot \frac{{S}_{S}^{E} \cdot {S}_{S}^{C}}{{S}_{S}^{T}}}{{ε}_{j}}$$

wherein, α is a weight, and 0<α<1.

Formulation of the neurological rehabilitation training program: integrating the patients whose degree of neurological injury ε_{J} is less than the standard cut-off value in the pelvic floor assessment into the neurological rehabilitation training, and formulating a passive auxiliary training with a total duration of 30 minutes and a total of 12 training templates, wherein one training template has a duration of 150 seconds and one training template contains 10 training actions, then, adjusting the proportion of fast contraction-fast release with contraction maintenance based on the scores for various functions of the fast muscle and the slow muscle, with the magnetic stimulation intensity being set as S_{M} in the pelvic floor assessment. If the score S_{F} for various functions of the fast muscle and the score S_{S} for various functions of the slow muscle are both lower than the standard cut-off value, the corresponding fast contraction-fast release or contraction maintenance is allocated 5, for example, 5 passive auxiliary fast contraction-fast release actions followed by 5 passive auxiliary contraction maintenance actions. If only one of the score S_{F} for various function of the fast muscle or the score S_{S} for slow muscle is lower than the standard cut-off value, the corresponding proportion of fast contraction-fast release action or contraction maintenance action increases by 10% every time the score lower by 10; the rest time between each action interval is at least 5 seconds.

Adjustment of the neurological rehabilitation training program: for patients engage in the neurological rehabilitation training, if the active-passive combination assessment score of the pelvic floor muscle increases in a training template, the magnetic stimulation intensity of the passive training decreases; if the active-passive combination assessment score of the pelvic floor muscle decreases in a training template, the intensity of magnetic stimulation increases. At the same time, if either the score for various function of the fast muscle or the score for various function of the slow muscle decreases in the scores for various function of the fast muscle and the slow muscle, the corresponding proportion of fast contraction-fast release action or contraction maintenance increases, on the contrary, the corresponding proportion decreases, if both scores decrease, the proportion remains unchanged. The adjustment of the training template for the neurological rehabilitation training can be achieved through the following method: calculating the active-passive combination assessment score of the pelvic floor muscle after each training template is completed, the magnetic stimulation intensity decreases by IT every time the score increases by 10, when the score exceeds 80, the magnetic stimulation intensity decreases to a minimum of IT and does not decrease any more; simultaneously calculating the scores for various functions of the fast muscle and the slow muscle, the corresponding proportion of fast contraction-fast release action or contraction maintenance action in a training template decreases by 10% every time the score for various functions of the fast muscle or the slow muscle increases by 10.

The formulation of the muscle rehabilitation training program, integrating the patients whose degree of neurological injury ε_{J} in pelvic floor assessment is higher than the standard cut-off value but whose active-passive combination assessment score M of the pelvic floor muscle is lower than the standard cut-off value into the muscle rehabilitation training, and formulating an active training with a total duration of 30 minutes and a total of 12 training templates, wherein one training template has a duration of 150 seconds and one training template contains 10 training actions, then, adjusting the proportion of fast contraction-fast release with contraction maintenance based on the scores for the fast and slow muscles. If the score S_{F} for various functions of the fast muscle and the score S_{S} for the slow muscle are both lower than the standard cut-off value, the corresponding fast contraction-fast release or contraction maintenance is allocated 5, for example, 5 passive fast contraction-fast release actions followed by 5 passive contraction maintenance actions. If only one of the score S_{F} for various function of the fast muscle or the score S_{S} for slow muscle is lower than the normal value, the corresponding proportion of fast contraction-fast release action or contraction maintenance action increases by 10%; the rest time between each action interval is at least 5 seconds;
the assessment of the pelvic floor muscle characteristic parameters during training, that is, immediately assessing the score for various function of the pelvic floor muscle after each template training is completed, one method for assessing the pelvic floor muscle characteristic parameters during training includes: for patients engage in the neurological rehabilitation training, calculating the various functional indexes of the fast and slow muscles and the active-passive combination assessment score of the pelvic floor muscle based on the passive contraction of the pelvic floor muscle during each magnetic stimulation auxiliary training. For patients engage in exercise function rehabilitation training, calculating the various functional indexes of the fast and slow muscles of the pelvic floor muscle, as well as the active-passive combination assessment score of the pelvic floor muscle in the training template for each fast contraction-fast release and contraction maintenance.

Adjustment of the muscle rehabilitation training program: for patients engage in the muscle exercise rehabilitation training, if the active-passive combination assessment score of the pelvic floor muscle increases after the training of a training template is completed, the amplitude of the template increases, on the contrary, it decreases. At the same time, either the score for various function of the fast muscle or the score for various function of the slow muscle decreases in the scores for various function of the fast muscle and the slow muscle, the corresponding proportion of fast contraction-fast release action or contraction maintenance increases, on the contrary, the corresponding proportion decreases. The adjustment of the training template for muscle exercise rehabilitation training can be achieved through the following method: calculating the active-passive combination assessment score of the pelvic floor muscle after each training template is completed, the amplitude of the template decreases by 10% every time the score increases by 10, when the score exceeds 80, the amplitude of the template increases to a maximum 80% and does not increase any more; on the contrary, it decreases to 30% and does not decrease any more. Simultaneously calculating the scores for the fast muscle and the slow muscle, the corresponding proportion of fast contraction-fast release action or contraction maintenance action in a training template decreases by 10% every time the score for the fast muscle or the slow muscle increases by 10.

It should be noted that in this article, relational terms such as first and second are only used to distinguish one entity or operation from another entity or operation, and do not necessarily require or imply any actual relationship or order between these entities or operations. Moreover, the terms “including”, “comprising”, or any other variation thereof are intended to encompass non-exclusive inclusion, such that a process, method, item or device that includes a series of elements includes not only those elements, but also includes other elements not explicitly listed, or also includes elements inherent to such process, method, item or device.

Finally, it should be noted that: the above description is only a preferred embodiment of the present disclosure and is not intended to limit the present disclosure. Although the present disclosure has been described in detail with reference to the aforementioned embodiments, those skilled in the art can still modify the technical solutions described in the aforementioned embodiments or equivalently replace some of the technical features. Any modifications, equivalent substitutions, improvements, etc. made within the spirit and principles of the present disclosure shall be included within the scope of protection of the present disclosure.

What is claimed is:

1. A non-contact pelvic floor assessment, characterized in that: the assessment includes the following steps:
S1, determining whether the user's sitting posture and force exerting way during a magnetic stimulation are correct based on a non-contact biofeedback, thereby obtaining the correct sitting posture and the correct force exerting way on the pelvic floor muscle;
S2, obtaining a non-contact feedback waveform based on the user using correct sitting posture and correct force exerting way on the pelvic floor muscle, assessing the maximum contraction force of the pelvic floor muscle fast muscle and the maximum contraction force of the pelvic floor muscle slow muscle based on the non-contact feedback waveform respectively, and generating a pelvic floor muscle fast muscle assessment template and a pelvic floor muscle slow muscle assessment template respectively;
S3, assessing various functions of the pelvic floor muscle fast muscle and the pelvic floor muscle slow muscle according to the pelvic floor muscle fast muscle assessment template and the pelvic floor muscle slow muscle assessment template;
S4, assessing the user's neuromuscular regulation function by means of active-passive combination, obtaining a corresponding score based on various functional indexes of the pelvic floor muscle fast muscle, various functional indexes of the pelvic floor muscle slow muscle and a degree of neurological injury, thereby calculating an integrate score for the active-passive combination assessment of the pelvic floor muscle.

2. The non-contact pelvic floor assessment of claim 1, characterized in that: in steps S1-S3, assessing various functions of the pelvic floor muscle fast muscle includes the following steps:
S2-1, collecting the non-contact feedback waveform for user to exert force using the pelvic floor muscle;
S2-2, setting a threshold grad* for the non-contact feedback waveform of the pelvic floor muscle according to the non-contact feedback database of the pelvic floor muscle, when the waveform is unimodal and the slope at the descent during relaxation is greater than grad*, it is determined that the user's sitting posture and force exerting way during the magnetic stimulation are correct; when the waveform is a bimodal wave and the slope at the descent during relaxation is small, it is determined that the force exerting way is to use the anal sphincter muscle for contraction, and the sitting posture and force exerting way are incorrect;
S2-3, using the maximum force to contract the pelvic floor muscle for x times rapidly when the user uses the correct sitting posture and the correct force exerting way on the pelvic floor muscle, thereby obtaining the non-contact feedback waveform;
wherein, x is a constant set by a system;
S2-4, assessing the maximum contraction force S_{Fmax} of the pelvic floor muscle fast muscle: the user uses the correct sitting posture and the correct force exerting way on the pelvic floor muscle, and uses the maximum force to rapidly contract the pelvic floor muscle for multiple time, according to the amplitude set D={D₁, D₂, . . . , D_{N}} of the non-contact feedback waveform, wherein the maximum contraction amplitude corresponds to the maximum distance Dₘₐₓ=max(D) that the pelvic floor muscle of the subject can contract, the maximum contraction force S_{Fmax} of the user's pelvic floor muscle is obtained correspondingly, the pelvic floor muscle fast muscle assessment template is generated based on the fast muscle assessment template database corresponding to the actual value of S_{Fmax};
S2-5, contracting the pelvic floor muscle for three times by user through using the correct sitting posture and the correct force exerting way on the pelvic floor muscle, the first pelvic floor muscle contraction force needs to reach a % of S_{Fmax}, the second pelvic floor muscle contraction force needs to reach b % of S_{Fmax}, and the third pelvic floor muscle contraction force needs to reach c % of S_{Fmax}, generating the fast muscle assessment template needing to contract the pelvic floor muscle for three times according to the fast muscle assessment template database corresponding to the actual value of the pelvic floor muscle contraction force;
the a, b and c are constants set by the system, and a<b<c;
S2-6, collecting the non-contact feedback waveform of the pelvic floor muscle contraction, and assessing a muscle strength
$${S}_{F}^{P}$$
of the user's pelvic muscle fast muscle according to the non-contact feedback waveform:
if S_{Fmax}<S_{std}, it indicates that the muscle strength
$${S}_{F}^{P}$$
of the user's fast muscle is weak, wherein S_{std} is a threshold for assessing the muscle strength of the fast muscle, which is set according to a database assessing big data;
S2-7, assessing a control force
$${S}_{F}^{C}$$
of the user's pelvic floor muscle fast muscle according to the non-contact feedback waveform, if a ratio N_{Fdown}/N_{F} of the number of data points N_{Fdown} in the lower portion of the template requirement line with the number of all data points N_{F} is >d %, it indicates that the user has poor control force
$${S}_{F}^{C}$$
on the fast muscle; if N_{Fdown}/N_{F}≤d %, it indicates that the user has normal control force
$${S}_{F}^{C}$$
on the fast muscle;
S2-8, assessing an explosive force
$${S}_{F}^{A}$$
of the user's pelvic floor muscle according to the non-contact feedback waveform: during a fast contraction-fast release, if
$${t}_{U P} \geq {t}_{UPstd}^{Ftop} ,$$
it indicates that the explosive force of the user's pelvic floor muscle is poor;
if
$$if {t}_{U P} < {t}_{UPstd}^{Ftop} ,$$
it indicates that the explosive force of the user's pelvic floor muscle is good;
wherein t_{UP} is a time taken to reach the peak of the amplitude,
$${t}_{UPstd}^{Ftop}$$
is a time cut-off value taken from rest to the highest point reached by the contraction during a fast contraction-fast release;
S2-9, assessing the fatigue level
$${S}_{F}^{T}$$
of the user's fast muscle according to the non-contact feedback waveform, if after contracting the pelvic floor muscle for multiple times, the non-contact feedback waveform is manifested as: during a fast contraction-fast release, if
$${t}_{D O W N} \geq {t}_{DOWNstd}^{Ftop} ,$$
it indicates that the user's pelvic floor muscle have entered into the fatigue period;
if
$$if {t}_{D O W N} < {t}_{DOWNstd}^{Ftop} ,$$
it indicated that the user's pelvic floor muscle have not yet entered into the fatigue period;
wherein t_{DOWN} is a time taken from the highest point to the lowest point of the amplitude in the waveform, and
$${t}_{DOWNstd}^{Ftop}$$
is a time cut-off value taken from the highest point reached by the contraction to rest during a fast contraction-fast release.

3. The non-contact pelvic floor assessment of claim 2, characterized in that: in steps S1-S3, assessing various functions of the pelvic floor muscle slow muscle includes the following steps:
S3-1, contracting the pelvic floor muscle for three times by user through using the correct sitting posture and the correct force exerting way on the pelvic floor muscle, contracting the pelvic floor muscle to enable the feedback amplitude to reach e % of the maximum contraction force S_{Fmax} of the pelvic floor muscle fast muscle and maintain it for a period of time f, then continuing to contract the pelvic floor muscle without relaxing to enable the feedback waveform to reach the amplitude as high as possible and maintain it for a period of time g, and taking an average value of the feedback amplitudes in g to represent the maximum contraction force S_{Smax} of the user's pelvic floor muscle slow muscle;
as high as possible refers to the user tries his best to contract to a certain amplitude and maintain it for a period of time while performing a contraction maintenance;
the e, f, and g are constants set by the system;
S3-2, generating a pelvic floor muscle slow muscle assessment template based on the maximum contraction force S_{Smax} of the pelvic floor muscle slow muscle;
S3-3, taking h % of the maximum contraction force S_{Smax} amplitude of the pelvic floor muscle as the template amplitude for assessment, contracting and maintain the pelvic floor muscle by the user through using the correct sitting posture and the correct force exerting way on the pelvic floor muscle;
the h is a constant set by the system;
S3-4, assessing an endurance
$${S}_{S}^{E}$$
of the user's pelvic floor muscle slow muscle according to the non-contact feedback waveform of the pelvic floor muscle contraction, if the height specified by the template cannot be reached by the contraction maintenance or the time for the contraction maintenance cannot reach the length of the contraction time of the threshold, or the non-contact feedback waveform occurs to deviate from the template to trend to decrease during the contraction maintenance phase, it indicates that the endurance
$${S}_{S}^{E}$$
of the slow muscle is weak, on the contrary, it indicates that the endurance of the slow muscle is good;
S3-5, assessing a control force
$${S}_{S}^{C}$$
of the user's pelvic floor muscle slow muscle according to the non-contact feedback waveform of the pelvic floor muscle contraction: if the waveform occurs to shake during contraction and relaxation, it is manifested on the non-contact feedback waveform as: if the ratio N_{Sdown}/N of the number of data points N_{Sdown} in the lower portion of the template requirement line with the number of all data points N is >d %, it indicates the user has poor control force
$${S}_{S}^{C}$$
on the slow muscle, if N_{Sdown}/N<d %, it indicates the user has good control force
$${S}_{S}^{C}$$
the slow muscle.

4. The non-contact pelvic floor assessment of claim 3, characterized in that: in step S4, the active-passive combination assessment of the pelvic floor muscle includes the following steps:
S4-1, the user sits in the correct posture on the magnetic stimulation therapy device equipped with a biofeedback module, gradually increasing the magnetic stimulation intensity and obtaining passive contraction feedback of the pelvic floor muscle under the magnetic field stimulation;
S4-2, stimulating the user's pelvic floor muscle starting from a magnetic stimulation intensity of x₂, when the stimulation intensity reaches intensity S_{M}, the biofeedback amplitude of passive contraction of the pelvic floor muscle no longer increases with the increase of magnetic stimulation intensity, it indicates that the pelvic floor muscle have reached the passive contraction threshold after reaching the magnetic stimulation intensity S_{M};
wherein, x₂ is a constant set by the system;
S4-3, assessing the neurological injury of the pelvic floor muscle and its degree of injury according to the non-contact feedback waveform of passive contraction of the pelvic floor muscle: comparing the magnetic stimulation intensity S_{M} with the standard cut-off value
$${S}_{M}^{std}$$
to determine whether there is the neurological injury of the pelvic floor muscle and its degree of injury ε_{J};
wherein,
$${ε}_{J} = \{ \begin{matrix}{x}_{3} + ( {S}_{M} - {S}_{M}^{std} ) / {S}_{M} , {S}_{M} > {S}_{M}^{std} \\ {x}_{4} + ( {S}_{M} - {S}_{M}^{std} ) / {S}_{M} , {S}_{M} < {S}_{M}^{std}\end{matrix}$$
wherein, x₃, x₄ are constants set by the system;
wherein, the standard cut-off value
$${S}_{M}^{std}$$
is a threshold which is generated based on a big data assessment database and is selected to distinguish the neurological injury of the pelvic floor muscle and its degree of injury;
S4-4, the calculation method for the integrate score M of the active-passive combination assessment of the pelvic floor muscle is:
$$M = \frac{α \cdot \frac{( {S}_{f}^{P} \cdot {S}_{F}^{C} \cdot {S}_{F}^{A} )}{{S}_{F}^{T}} + ( 1 - α ) \cdot \frac{{S}_{S}^{E} \cdot {S}_{S}^{C}}{{S}_{S}^{T}}}{{ε}_{J}}$$
wherein, α is a weight, and 0<α<1;
$${S}_{F}^{P}$$
indicates the muscle strength of the fast muscle,
$${S}_{F}^{C}$$
indicates the control force of the fast muscle,
$${S}_{F}^{A}$$
indicates the explosive force of the fast muscle,
$${S}_{F}^{T}$$
indicates the fatigue level of the fast muscle,
$${S}_{S}^{E}$$
indicates the endurance of the slow muscle,
$${S}_{S}^{C}$$
indicates the control force of the slow muscle,
$${S}_{S}^{T}$$
indicates the fatigue level of the slow muscle, ε_{J} indicates the degree of neurological injury.

5. The non-contact pelvic floor assessment of claim 1, characterized in that: in step S4, the active-passive combination mode refers to:
the active mode is that the user contracts the pelvic floor muscle actively to obtain various indexes of the fast muscle and slow muscle of the pelvic floor muscle; the passive mode is the passive contraction feedback of the user's pelvic floor muscle under the magnetic field stimulation, to determine the existence of the user's pelvic floor muscle neurological injury and the degree of injury.

6. A non-contact pelvic floor training method, characterized in that: the training method comprises the following steps:
S1, formulating a rehabilitation training program, which includes a neurological rehabilitation training program and a muscle rehabilitation training program;
S2, assessing pelvic floor muscle characteristic parameters during the rehabilitation training, and calculating a score for various functions of the pelvic floor muscle;
S3, making a real-time adjustment to the training program based on changes in the assessment results of the pelvic floor muscle.

7. The non-contact pelvic floor training method of claim 6, characterized in that: in step S1, the method of formulating a rehabilitation training program includes the following steps:
S7-1, integrating users whose magnetic stimulation intensity S_{M} in pelvic floor assessment is less than the corresponding magnetic stimulation intensity standard cut-off value
$${S}_{M}^{std}$$
into the neurological rehabilitation training, and formulating a passive auxiliary training with a total of k minutes for them, and there are m training templates in total, each training template has a duration of n, each training template contains p training actions;
the k, n, and p are constants set by the system;
the active training refers to the user performs two types of actions that are fast contraction-fast release and contraction maintenance actively according to the prompts on the display, the passive auxiliary training refers to the user performs the fast contraction-fast release action or the contraction maintenance action actively while applying the magnetic stimulation to assist and thereby to strengthen the actions;
S7-2, adjusting the proportion of fast contraction-fast release with contraction maintenance based on a score for various functions of the fast muscle and a score for various functions of the slow muscle, with the magnetic stimulation intensity being S_{M};
setting the standard cut-off values for the score for various functions of the fast muscle and the slow muscle according to the big data classification sample, and establishing the standard cut-off value set S_{FStd} for the score S_{F} for various functions of the fast muscle, the standard cut-off value set S_{FStd} includes the standard cut-off value
$${S}_{Fstd}^{P}$$
for the muscle strength of the fast muscle, the standard cut-off value
$${S}_{Fstd}^{C}$$
for the control force of the fast muscle, the standard cut-off value
$${S}_{Fstd}^{A}$$
for the explosive force of the fast muscle, and the standard cut-off value
$${S}_{Fstd}^{T}$$
for the fatigue level of the fast muscle, establishing the standard cut-off value set S_{Sstd} for the score Sₛ for various functions of the slow muscle, the standard cut-off value set S_{Sstd} includes the standard cut-off value
$${S}_{Fstd}^{E}$$
for the endurance of the slow muscle, the standard cut-off value
$${S}_{Fstd}^{C}$$
for the control force of the slow muscle and the standard cutoff value
$${S}_{Fstd}^{T}$$
for the fatigue level of the slow muscle;
if the score S_{F} for various functions of the fast muscle and the score S_{S} for various functions of the slow muscle are both lower than the corresponding standard cut-off values in the standard cut-off value sets S_{FStd}, S_{Sstd}, the corresponding fast contraction-fast release or contraction maintenance is allocated q; wherein, S_{F} includes
$${S}_{F}^{P} , {S}_{F}^{C} , {S}_{F}^{A} and {S}_{F}^{T} ,$$
Sₛ includes
$${S}_{S}^{E} , {S}_{S}^{C} and {S}_{S}^{T} ;$$
if only one of the score S_{F} for various functions of the fast muscle or the score S_{S} for various functions of the slow muscle is lower than the corresponding standard cut-off value in the corresponding standard cut-off value sets S_{Fstd}, S_{std}, the corresponding proportion of fast contraction-fast release action or contraction maintenance action increases by z % every time the score decreases by y, and the rest time between each action interval is at least r;
the q, y, z and r are constants set by the system;
S7-3, setting the cut-off value M_{std} for the active-passive combination assessment score of the pelvic floor muscle according to the big data classification sample, setting the standard cut-off value ε_{std} for the degree of neurological injury, and obtaining the degree of neurological injury ε_{J}; if the degree of neurological injury ε_{J} is higher than ε_{std}, it indicates that the nerve is not injured; if the degree of neurological injury is lower than ε_{std}, it indicates the presence of neurological injury, and the smaller the value, the more severe the degree of injury; integrating the users whose degree of neurological injury ε_{J} in the pelvic floor assessment is higher than the standard cut-off value ε_{std} for the degree of neurological injury but whose active-passive combination assessment score M of the pelvic floor muscle is lower than its corresponding standard cut-off value M_{std} into the muscle rehabilitation training, formulating an active training program with a total duration of k₁ and m₁ training templates, wherein each training template has a duration of n₁ and each training template contains p₁ training actions;
the k₁, m₁, n₁ and p₁ are constants set by the system;
if the score S_{F} for various functions of the fast muscle and the score S_{S} for various functions of the slow muscle are both lower than the corresponding standard cut-off values in the standard cut-off value sets S_{FStd}, S_{Sstd} of the fast and slow muscle, the corresponding fast contraction-fast release or contraction maintenance are allocated q₁;
if only one of the score S_{F} for various functions of the fast muscle or the score S_{S} for various functions of the slow muscle is lower than the normal value, the corresponding proportion of fast contraction-fast release action or contraction maintenance action increases by z₁%, and the rest time between each action interval is at least r₁;
the q₁, z₁, and r₁ are constants set by the system.

8. The non-contact pelvic floor training method of claim 6, characterized in that: in steps S2-S3, the method of making a real-time adjustment to the training program includes the following steps:
S8-1, when the user engages in neurological rehabilitation training, calculating the score for various functions of the fast muscle, the score for various functions of the slow muscle, and active-passive combination assessment score of the pelvic floor muscle based on the passive contraction of the pelvic floor muscle during each magnetic stimulation auxiliary training; when the user engages in muscle rehabilitation training, calculating the score for various functions of the fast muscle, the score for various functions of the slow muscle, and active-passive combination assessment score of the pelvic floor muscle based on the training template in each fast contraction-fast release and contraction maintenance;
S8-2, when the user engages in neurological rehabilitation training:
if the active-passive combination assessment score of the pelvic floor muscle increases after the training of each training template is completed, the magnetic stimulation intensity decreases;
if the active-passive combination assessment score of the pelvic floor muscle decreases after the training of each training template is completed, the magnetic stimulation intensity increases;
if either score in
$${S}_{F}^{P} , {S}_{F}^{C} , {S}_{F}^{A} , {S}_{F}^{T} , {S}_{S}^{E} , {S}_{S}^{C} and {S}_{S}^{T}$$
decreases, the proportion of fast contraction-fast release or contraction maintenance increases correspondingly, and on the contrary, the corresponding proportion of fast contraction-fast release or contraction maintenance decreases;
if the scores of the fast muscle and the slow muscle decrease in the scores
$${S}_{F}^{P} , {S}_{F}^{C} , {S}_{F}^{A} , {S}_{F}^{T}$$
for various functions of the fast muscle and the scores
$${S}_{S}^{E} , {S}_{S}^{C} , {S}_{S}^{T}$$
for various functions of the slow muscle, the proportion remains unchanged;
S8-3, calculating the active-passive combination assessment score of the pelvic floor muscle after the training of each training template is completed, the magnetic stimulation intensity u decreases every time the score increases by s₁, when the score exceeds s₂, the magnetic stimulation intensity decreases to be u₀, and does not decrease any more; calculating various functions of the fast and slow muscles, when each function of the fast muscle or the slow muscle rises s₃, the corresponding proportion of fast contraction-fast release action or contraction maintenance action in one training template decreases by v %;
the s₁, u, s₂, u₀, s₃ and v are constants set by the system;
S8-4, when the user engages in muscle rehabilitation training:
if the active-passive combination assessment score of the pelvic floor muscle increases after the training of each training template is completed, the amplitude of the template increases;
if the active-passive combination assessment score of the pelvic floor muscle decreases after the training of each training template is completed, the amplitude of the template decreases;
if one of the score for various functions of the fast muscle or the score for various functions of the slow muscle decreases in the score for various functions of the fast muscle and the score for various functions of the slow muscle, the proportion of fast contraction-fast release or contraction maintenance increases correspondingly, on the contrary, the corresponding proportion decreases;
S8-5, calculating the active-passive combination assessment score of the pelvic floor muscle after each training template is completed, the template amplitude decreases by v₁% every time the score increases by s₄, when the score exceeds s₅, the template amplitude increases to the highest v₂% and does not increase any more;
on the contrary, the template amplitude decreases to v₃% and does not decrease any more;
calculating the scores for various functions of the fast and slow muscles, when the score for various functions of the fast muscle or the score for various functions of the slow muscle increases by s₅, the corresponding proportion of fast contraction-fast release action or contraction maintenance action in one training template decreases by v₄%;
the s₄, v₁, s₅, v₂, v₃ and v₄ are constants set by the system.
